(19) United States
(12) Patent Application Publication
(10) Pub. No.: US 2012/0190991 A1
(43) Pub. Date: Jul. 26, 2012

(54) System and Method for Detecting a Clinically-Significant Pulmonary Fluid Accumulation Using an Implantable Medical Device

(75) Inventors: Gene A. Bornzin, Simi Valley, CA (US); Steve Koh, South Pasadena, CA (US); James S. Whiting, Los Angeles, CA (US); Neal L. Eigler, Malibu, CA (US); Brian M. Mann, Edgartown, MA (US)

(73) Assignee: PACESETTER, INC., Sylmar, CA (US)

(21) Appl. No.: 13/012,595

(22) Filed: Jan. 24, 2011

(51) Int. Cl. A61B 5/021

(52) U.S. Cl. 600/485

(57) ABSTRACT

Techniques are provided for detecting a clinically-significant pulmonary fluid accumulation within a patient using a pacemaker or other implantable medical device. Briefly, the device detects left atrial pressure (LAP) within the patient and tracks changes in the LAP values over time that are indicative of possible pulmonary fluid accumulation within the patient. The device determines whether the changes in LAP values are sufficiently elevated and prolonged to warrant clinical intervention using, e.g., a predictor model-based technique. If the fluid accumulation is clinically significant, the device then generates warning signals, records diagnostics, controls therapy and/or titrates diuretics. False positive detections of pulmonary edema due to transients in LAP are avoided with this technique. Pulmonary artery pressure (PAP)-based techniques are also described.

### FIELD OF THE INVENTION

The invention generally relates to implantable medical devices, such as pacemakers and implantable cardioverter/defibrillators (ICDs), and in particular to techniques for monitoring pulmonary fluid levels within patients using such devices.

### BACKGROUND OF THE INVENTION

Heart failure is a debilitating disease in which abnormal function of the heart leads in the direction of inadequate blood flow to fulfill the needs of the tissues and organs of the body. Typically, the heart loses propulsive power because the cardiac muscle loses capacity to stretch and contract. Often, the ventricles do not adequately eject or fill with blood between heartbeats and the valves regulating blood flow become leaky, allowing regurgitation or back-flow of blood. The impairment of arterial circulation deprives vital organs of oxygen and nutrients. Fatigue, weakness and the inability to carry out daily tasks may result. Not all heart failure patients suffer debilitating symptoms immediately. Some may live actively for years. Yet, with few exceptions, the disease is relentlessly progressive. As heart failure progresses, it tends to become increasingly difficult to manage. Even the compensatory responses it triggers in the body may themselves eventually complicate the clinical prognosis. For example, when the heart attempts to compensate for reduced cardiac output, it adds muscle causing the ventricles (particularly the left ventricle) to grow in volume in an attempt to pump more blood with each heartbeat. This places a still higher demand on the heart's oxygen supply. If the oxygen supply falls short of the growing demand, as it often does, further injury to the heart may result. The additional muscle mass may also stiffen the heart walls to hamper rather than assist in providing cardiac output. A particularly severe form of heart failure is congestive heart failure (CHF) wherein the weak pumping of the heart leads to build-up of fluids in the lungs and other organs and tissues.

Pulmonary edema is a swelling and/or fluid accumulation in the lungs often caused by heart failure. Briefly, the poor cardiac function resulting from heart failure can cause blood to back up in the lungs, thereby increasing blood pressure in the lungs, particularly pulmonary venous pressure. The increased pressure pushes fluid—but not blood cells—out of the blood vessels and into lung tissue and air sacs (i.e. the alveoli). This can cause severe respiratory problems and, left untreated, can be fatal. Pulmonary edema can also arise due to other factors besides heart failure, such as infections.

In view of the potential severity of pulmonary edema, it is highly desirable to detect the fluid accumulations associated with pulmonary edema so that appropriate therapy can be provided. Many patients susceptible to pulmonary edema are candidates for pacemakers, ICDs or other implantable medical devices. Accordingly, it is desirable to provide such devices with the capability to automatically detect and respond to pulmonary fluid accumulations, and aspects of the invention are generally detected to that end.

One promising technique for monitoring pulmonary congestion arising from heart failure uses left atrial pressure (LAP) measurements. In this regard, fluid retention leads to elevation in blood volume that leads to increases in left heart filling pressures (and hence increased LAP) which in turn leads to elevation of pulmonary arterial and pulmonary venous pressures. Elevated pulmonary venous pressure increases pulmonary capillary hydrostatic pressure, P_{cap}. Excessive increases in pulmonary capillary pressure can result in fluid transudation into the alveoli of the lung. This interferes with gas exchange. The patient then accumulates carbon dioxide and, even more distressing, the patient becomes hypoxic. The hypoxia causes severe distress in the heart failure patient and, if untreated, can be fatal.

Fluid normally tends to stay in the vascular system and not transude through the capillary membrane into the alveolar space. This is because of a balance of hydrostatic pressure and fluid oncotic pressures that maintain a gradient, which maintains fluid in the circulation. Hydrostatic pressure is due to mechanical pressure in the blood or tissue. Oncotic pressure is associated with colloidal particles that create an osmotic pressure because the particles do not diffuse across membrane barriers. In the blood, the primary contributor to oncotic pressure is plasma albumin, which is a blood protein. The effective pressure gradient drives water into the alveoli is ΔP. P_{cap} is the pulmonary capillary pressure that forces water into the alveoli; Pᵢₛ is the pressure on the inside of alveoli (usually negative due to the process of breathing). Note that the hydrostatic pressure gradient across the between the capillary and the alveolar cavity is P_{cap}−Pᵢₛ. Similarly, there is an oncotic pressure gradient across the membrane. π_{cap} is the oncotic pressure of the capillary blood. It is primarily due to blood proteins (primarily albumin) that create colloid oncotic pressure in blood plasma. πᵢₛ is the colloid oncotic pressure of the fluid inside the alveoli.

Equation 1 represents the difference in pressure gradients across membranes that separate the blood in the capillary to the fluid inside the alveoli. If ΔP is positive, then fluid will migrate into the alveoli space and cause pulmonary edema.

ΔP=(P_{cap}−Pᵢₛ)−(π_{cap}−πᵢₛ) (1)

P_{cap}=26 mm Hg

Pᵢₛ≈−2 to −5 mmHg

π_{cap}≈24 mmHg

πᵢₛ≈0 to 5 mmHg (2)

ΔP=(26−5)−(24−3)=0 mmHg (3)

Equation 2 provides typical values for these pressures. In Equation 3, above, the gradient is 0 mmHg. Under these conditions, there is a precise balance between the hydrostatic pressure and the oncotic pressures and theoretically fluid should not transude out of the lung capillaries into the lung alveoli to cause pulmonary edema. In general, the lungs operate with a relatively large negative gradient, ΔP, of about −12 mmHg because the typical pulmonary capillary pressure, P_{cap}, is about 14 mmHg. Under these conditions, fluid balance stays in favor of the blood and the lungs remain free of excess fluid. Equation 4 represents normal conditions:

ΔP=(14−5)−(24−3)=−12 mmHg (4)

In heart failure, however, there is a tendency for the pulmonary venous and arterial pressure to elevate. This is reflected in left atrial pressures in excess of 26 mmHg. The pulmonary capillary pressures also reach similar levels. When this occurs, fluid can transude into lungs and in particular into the alveoli. This constitutes pulmonary edema and, if left to progress to significant levels, gas exchange in the lungs can be severely hampered and the patient effectively suffocates because the lungs become filled with fluid. This process may take place over tens of minutes to hours or even days depending primarily on the pressure gradient ΔP. Equation 5 provides typical values for these pressures during pulmonary edema.

ΔP=(36−5)−(24−3)=10 mmHg (5)

In view of the foregoing, LAP measurements can theoretically be used by an implantable device to detect pressure gradients leading to potentially dangerous levels of pulmonary congestion. For example, if LAP is found to exceed the critical threshold of 26 mmHg, this may be indicative of excessive pulmonary fluid accumulation for which clinical intervention might be warranted. However, it has been found that transients in LAP, which are reflected in elevations of the pulmonary capillary pressure, often briefly exceed the critical level of 26 mmHg. (This observation regarding LAP transients is not necessarily recognized in the prior art. Indeed, no admission is made herein that any of the insights or analysis provided in this Background section necessarily constitute prior art to the claimed invention.) If LAP happens to be sampled by the implantable device during one of these transients, this might lead to a diagnosis of elevated LAP and necessitate making a clinical decision to treat the elevated pressure with a diuretic. In fact, brief transients in the LAP to relatively high levels are often not relevant because these episodes may not persist long enough to create any clinically relevant level of pulmonary edema. Responding to these transient elevations by treating the patient with diuretics is not only unnecessary but can be dangerous because the patient might become dehydrated if excess drugs are given. Similar problems might arise with pulmonary artery pressure measurements.

Accordingly, it would be desirable to provide techniques for detecting clinically-significant pulmonary fluid accumulations within patients and it is to this end that the invention is primarily directed.

### SUMMARY OF THE INVENTION

In accordance with an exemplary embodiment of the invention, techniques are provided for detecting clinically-significant pulmonary fluid accumulations within a patient in which an implantable medical device is implanted. In one example, the device detects values representative of left atrial pressure (LAP) within the patient using an LAP transducer or other suitable LAP sensing system, technique or proxy. The device tracks changes in the LAP values over time indicative of possible pulmonary fluid accumulation within the patient. The device then determines whether the changes in LAP values are sufficiently elevated and prolonged to warrant clinical intervention. Thereafter, at least one device function is controlled in response to a determination that clinical intervention is warranted, such as generating warning signals, recording diagnostics, controlling therapy and/or titrating diuretics. Thus, at least some aspects of the invention recognize the aforementioned problem with LAP transients and provide techniques for identifying clinically-significant pulmonary fluid accumulations despite such transients.

In an illustrative embodiment, a predictive model is applied to the detected LAP values to estimate or “predict” lung fluid volumes based on transfer functions. In an example where the implantable device is a pacemaker equipped with pacing/sensing leads and a LAP transducer, LAP is measured periodically using the transducer every few minutes (e.g. every five minutes) or hours. A transfer function is applied to the LAP values to generate values representative of pulmonary fluid accumulation (ΔV). The predicted ΔV values are compared against fluid accumulation thresholds indicative of a clinically-significant sustained accumulations, such as a threshold in the range of 18 mmHg to 26 mmHg. The transfer function relating LAP to ΔV may be represented by:

LAP→k/(τ·s+1)→ΔV (6)

where τ is representative of an exponential time parameter and s is a complex variable as used in Laplace transforms and k is a constant and wherein is dependent on posture. That is, τ can be represented by a τᵤₚ value indicative of an exponential rate of change while LAP is increasing and a τ_{down} value representative of an exponential rate of change while LAP is decreasing. Changes in LAP can be caused by a change in posture from supine to a standing posture. In this regard, when a patient is standing, sitting or walking, LAP is usually low (typically only about 5 mmHg.) When the patient is supine and inactive, LAP is usually higher (typically about 15 mmHg.) Of course, other factors alter LAP in addition to posture including cardiac function, salt intake increasing fluid retention, and pharmaceutical interventions. Typically, when working with Laplace transforms τ does not vary but for this discussion we will assert that the time constant, τ, is actually dependent on whether LAP is increasing or decreasing. Since the solution of this transfer function is usually performed digitally, adding direction sensitivity to τ straight forward.

Based on pre-determined calibrated values for k and τ for the patient, the device applies detected values for LAP to the transfer function to yield approximated or predicted values for ΔV for comparison against the threshold. Values for k and τ can be determined for the patient (i.e. calibrated) by, e.g., measuring ranges of time-varying values for LAP and ΔV within the patient following various changes in posture between supine and standing positions and then deriving suitable values for k and τ from the measure values. The values for ΔV for use in calibrating the predictor model may be determined, e.g., by using a suitable external pulmonary fluid detection system or by using an internal pulmonary fluid detection or proxy, if so equipped. The transfer function relating LAP to ΔV can be regarded as providing a low-pass filter that smoothes LAP values into filtered values for threshold comparison. This smoothing eliminates the transients that might otherwise cause false positives.

In some implementations, a drug pump is implanted within the patient and provided with suitable diuretics. The pacemaker directly controls the delivery and titration of the diuretics based on whether there is a clinically-significant pulmonary fluid accumulation in the lungs. In other implementations, the pacemaker transmits information to an external device (such as a bedside monitor or hand-held interface device) for notifying the patient or caregiver of the need to adjust the dosage of diuretics or other medications.

In other examples, pulmonary artery pressure (PAP) is used instead of LAP.

System and method examples of the invention are described herein.

### BRIEF DESCRIPTION OF THE DRAWINGS

The above and further features, advantages and benefits of the invention will be apparent upon consideration of the present description taken in conjunction with the accompanying drawings, in which:

FIG. 1 illustrates pertinent components of an implantable medical system having a pacemaker or ICD capable of detecting a clinically-significant pulmonary fluid accumulation within a patient using, in one example, a predictor model;

FIG. 2 is a flowchart providing an overview of the pulmonary fluid evaluation technique for detecting a clinically-significant pulmonary fluid accumulation based on LAP, which may be performed by the system of FIG. 1;

FIG. 3 is a block diagram illustrating a lung fluid predictor model, which may be exploited in accordance with the general technique of FIG. 2 to detect a clinically-significant pulmonary fluid accumulation;

FIG. 4 is a flowchart summarizing the use of the lung fluid predictor model of FIG. 3;

FIG. 5 is a flowchart that more fully illustrates a set of transfer functions for use the use of the lung fluid predictor model of FIG. 3;

FIG. 6 is a flowchart illustrating the predictor model-based technique of FIG. 3 in greater detail;

FIG. 7 is a graph illustrating variations in average LAP, smoothed pressure and predictor model output signals, which are generated or exploited by the techniques of FIGS. 3-6;

FIG. 8 is a graph illustrating just the variations in predictor model output signals of FIG. 7, which are generated or exploited by the technique of FIG. 6;

FIG. 9 is a flowchart providing a calibration technique for determining values for transfer function parameters relating posture, LAP and pulmonary fluids, for use with the technique of FIG. 6;

FIG. 10 is a flowchart providing an overview of the pulmonary fluid evaluation technique for detecting a clinically-significant pulmonary fluid accumulation based on PAP, which may be performed by the system of FIG. 1;

FIG. 11 is a simplified, partly cutaway view, illustrating the pacer/ICD of FIG. 1 along with at set of leads implanted into the heart of the patient; and

FIG. 12 is a functional block diagram of the pacer/ICD of FIG. 11, illustrating basic circuit elements that provide cardioversion, defibrillation and/or pacing stimulation in the heart and particularly illustrating components for evaluating pulmonary fluid accumulations using the techniques of FIGS. 2-10.

### DETAILED DESCRIPTION OF THE PREFERRED EMBODIMENTS

The following description includes the best mode presently contemplated for practicing the invention. This description is not to be taken in a limiting sense but is made merely to describe general principles of the invention. The scope of the invention should be ascertained with reference to the issued claims. In the description of the invention that follows, like numerals or reference designators will be used to refer to like parts or elements throughout.

### Overview of Implantable System

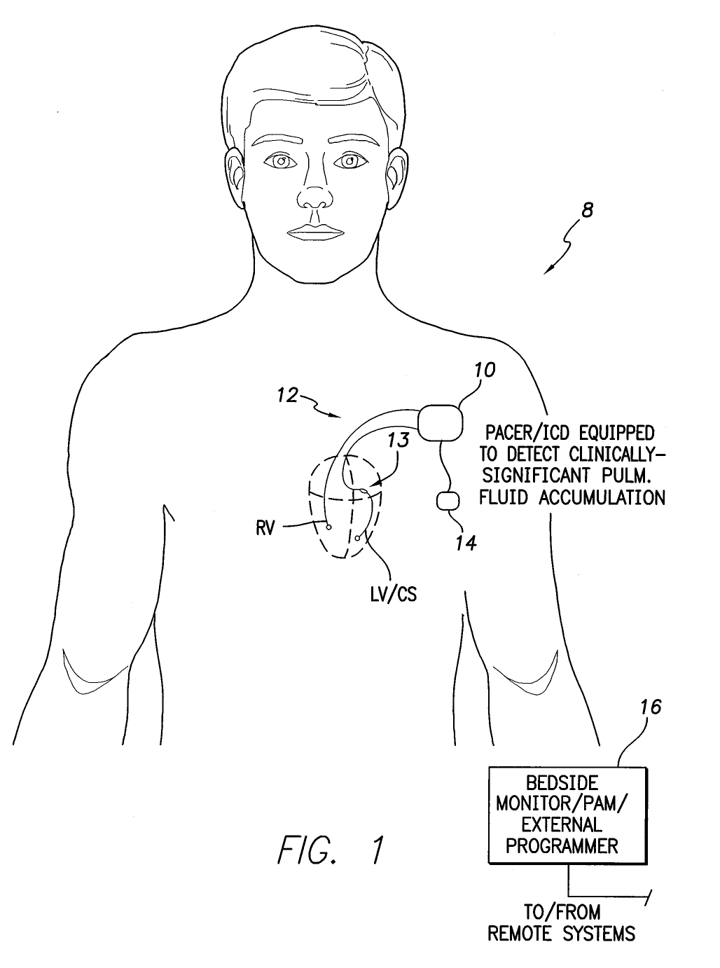

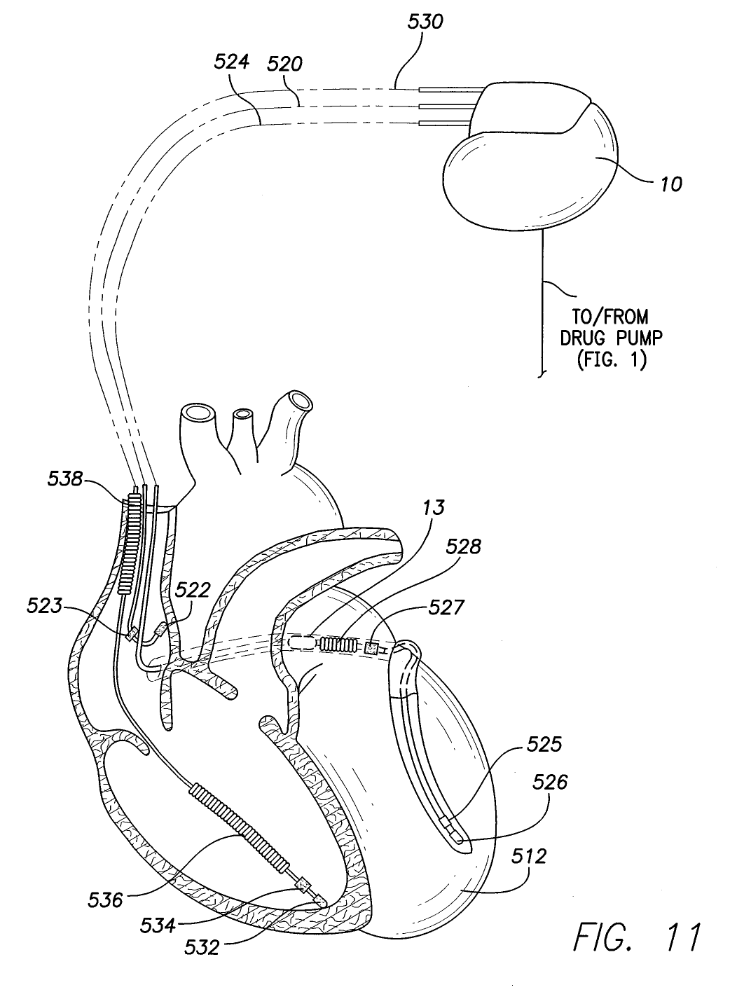

FIG. 1 illustrates an implantable medical system 8 capable of detecting a clinically-significant pulmonary fluid accumulation based on trends in LAP measurements and/or PAP measurements. The system is further capable of titrating dosages of diuretics or other medications in response, as well as performing various other therapeutic or diagnostic functions. To these ends, medical system 8 includes a pacer/ICD 10 or other cardiac rhythm management device equipped with one or more cardiac sensing/pacing leads 12 implanted within the heart of the patient. In FIG. 1, only two exemplary leads are shown: an RV lead and an LV lead implanted via the coronary sinus (CS). A more complete set of leads is illustrated in FIG. 11.

An LAP transducer sensor 13 is shown mounted to the LV/CS lead on or near the left atrium for sensing LAP. This particular LAP sensing configuration is merely illustrative. More information regarding LAP sensors and suitable locations for mounting such sensors are discussed in, for example, U.S. Published Patent Application 2003/0055345 of Eigler et al., entitled “Permanently Implantable System and Method for Detecting, Diagnosing and Treating Congestive Heart Failure.” See, also, U.S. patent application Ser. No. 11/856,443, filed Sep. 17, 2007, of Zhao et al., entitled “MEMS-Based Left Atrial Pressure Sensor for use with an Implantable Medical Device.” (A07P1145) PAP sensors for use with a PAP-based implementation are discussed below.

In examples where the system is intended to automatically titrate diuretics based on pulmonary fluid accumulations, an implanted or subcutaneous drug pump or other drug dispensing device 14 may be used, which is controlled by the pacer/ICD. Implantable drug pumps for use in dispensing medications are discussed in U.S. Pat. No. 5,328,460 to Lord, et al., entitled “Implantable Medication Infusion Pump Including Self-Contained Acoustic Fault Detection Apparatus.”

In other embodiments, information pertaining to any clinically-significant pulmonary fluid accumulation is transmitted to an external system 16, which generates diagnostic displays instructing the patient to take certain dosages of diuretics or other medications. System 16 may include, for example, an external programmer, bedside monitor or hand-held personal advisory module (PAM). Data from the external system can be forwarded to a centralized system such as the Merlin.Net system, the HouseCall™ remote monitoring system or the Merlin@home systems of St. Jude Medical for notifying the clinician of a clinically-significant pulmonary fluid accumulation.

Warnings as to a clinically-significant pulmonary fluid accumulation may also be generated using the bedside monitor, PAM, or an internal warning device provided within the pacer/ICD. The internal warning device (which may be part of pacer/ICD) can be a vibrating device or a “tickle” voltage device that, in either case, provides perceptible stimulation to the patient to alert the patient. The bedside monitor or PAM can provide audible or visual alarm signals to alert the patient or caregiver, as well as any appropriate textual or graphic displays. In addition, diagnostic information pertaining to changes in pulmonary fluid levels (and to any medical conditions detected therefrom) may be stored within the pacer/ICD for subsequent transmission to an external programmer (not shown in FIG. 1) for review by a clinician during a follow-up session between patient and clinician. The clinician then prescribes any other appropriate therapies to address the condition. The clinician may also adjust the operation of the pacer/ICD to activate, deactivate or otherwise control any therapies provided.

Additionally, the pacer/ICD performs a wide variety of pacing and/or defibrillation functions such as delivering pacing in response to an arrhythmia or generating and delivering shocks in response to fibrillation. Also, in some examples, the device is equipped to deliver cardiac resynchronization therapy (CRT). Briefly, CRT seeks to normalize asynchronous cardiac electrical activation and resultant asynchronous contractions associated with CHF by delivering synchronized pacing stimulus to both ventricles. The stimulus is synchronized so as to improve overall cardiac function. This may have the additional beneficial effect of reducing the susceptibility to life-threatening tachyarrhythmias.

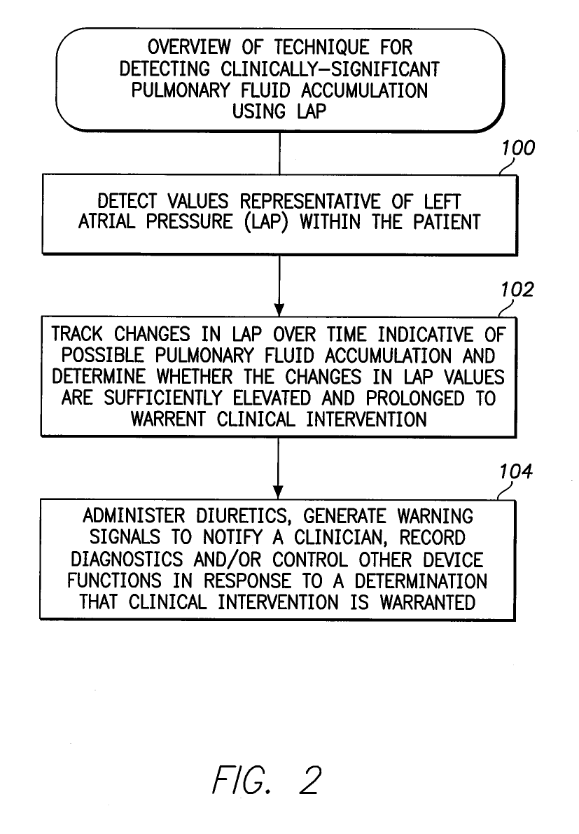

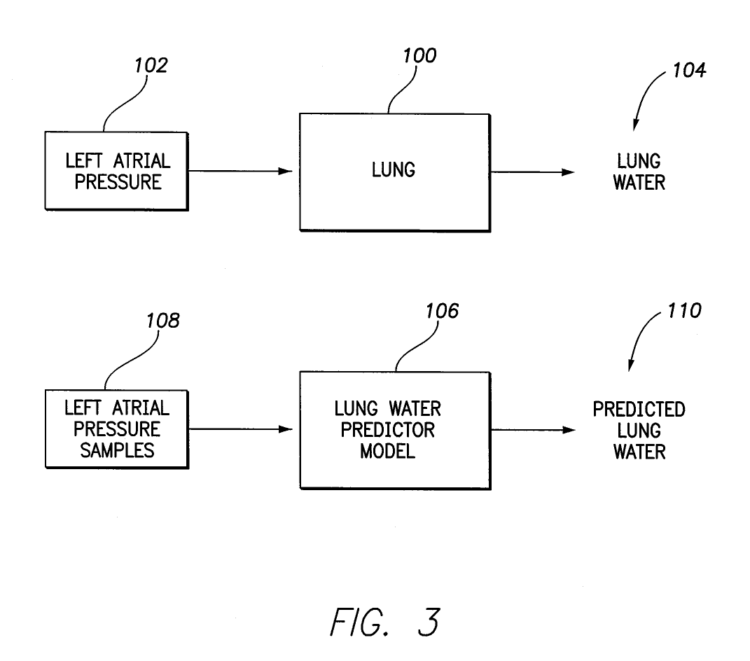

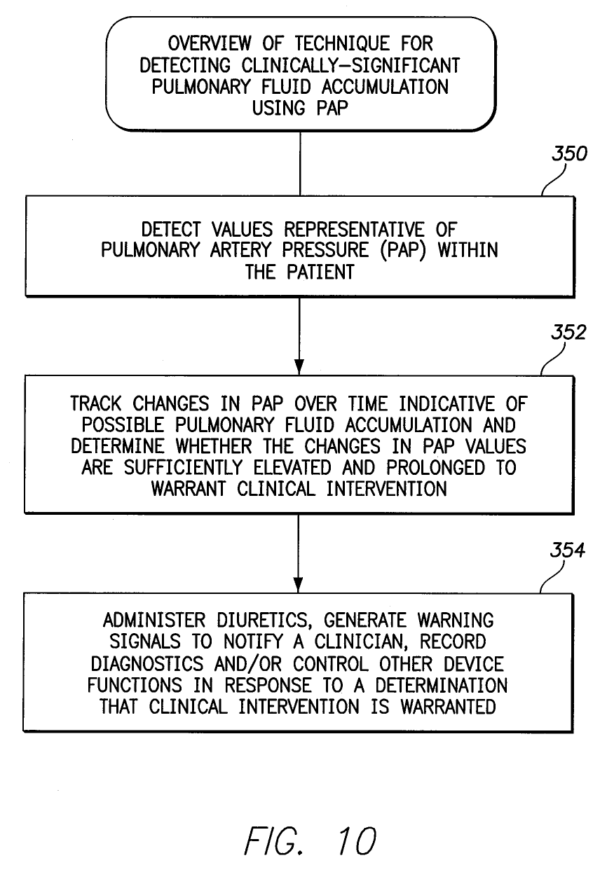

FIG. 2 broadly summarizes a general technique for detecting a clinically-significant pulmonary fluid accumulation based on LAP that may be exploited by the pacer/ICD of FIG. 1 or other suitably equipped systems. Beginning at step 100, the pacer/ICD detects values representative of LAP within the patient and then, at step 102, tracks changes in LAP over time indicative of possible pulmonary fluid accumulation and determines whether the changes in LAP values are both sufficiently elevated and sufficiently prolonged to warrant clinical intervention. That is, at least part of the determination of whether the pulmonary fluid accumulation is clinically-significant is made based on an elevation in LAP in combination with the duration with which LAP remains elevated, thereby avoiding false positive event detections due to LAP transients. In one example, described below with reference to FIGS. 3-9, a predictor model is employed to process LAP values to estimate pulmonary fluid volumes to make this determination. At step 104, the pacer/ICD then administers diuretics, generates warning signals to notify the clinician, records diagnostics and/or controls other device functions in response to a determination that clinical intervention is warranted. At similar technique for use with PAP is shown in FIG. 10, discussed below.

Hence, FIGS. 1 and 2 provide an overview of an implantable system and method capable of detecting a clinically-significant pulmonary fluid accumulation and further capable of titrating diuretics in response thereto or controlling other forms of therapy and for delivering appropriate warnings, if needed. Embodiments may be implemented that do not necessarily perform all of these functions. Rather, embodiments may be implemented that provide, for example, only for detecting a clinically-significant pulmonary fluid accumulation and generating diagnostic information for clinician review.

Also note that systems provided in accordance with the invention need not include all the components shown in FIG. 1. In many cases, for example, the implantable system will include only the pacer/ICD and its leads. Drug pumps are not necessarily employed. Some implementations may employ an external monitor for generating warning signals but no internal warning device. These are just a few exemplary embodiments. No attempt is made herein to describe all possible combinations of components that may be provided in accordance with the general principles of the invention. Also, note that, although internal signal transmission lines are shown in FIG. 1 for interconnecting implanted components, wireless signal transmission might alternatively be employed. In addition, it should be understood that the particular shape, size and locations of the implanted components shown in FIG. 1 are merely illustrative and may not necessarily correspond to actual implant locations. In particular, preferred implant locations for the leads are more precisely illustrated in FIG. 11.

### Predictor Model-based Technique

FIGS. 3-6 illustrate an exemplary technique for detecting a clinically-significant pulmonary fluid accumulation using a predictor model. Before describing an exemplary predictor model and the techniques with which the pacer/ICD exploits the model, the following more general observations are provided as background for the use of the model. Further to the analysis provided in the Background above, one can picture that the rate of change of alveolar water is related to passive transport across the alveolar membrane driven by hydrostatic pressure and oncotic gradients. During the process of exudation into the alveolar space, sodium ions enter the pulmonary alveolar space raising the alveolar πᵢₛ, which tends to increase the balance of water into the alveolar space. To counteract the exudation of lung water into the alveolar space, membrane-based pumps actively transport sodium out of the alveolar space in order to restore the πᵢₛ normal levels. Once the capillary pressure decreases to normal levels, the active transport of sodium out of the alveolar space reduces lung water to normal levels. Resolution of pulmonary edema is due to active transport of sodium and by passive diffusion due to a reduced hydrostatic pressure gradient.

Equation 7 describes the processes that take place to change the volume of lung water, V_{H2O}. Here, kₚ is the passive transport constant, which describes the volume rate that water moves across the alveolar membrane as the capillary pressure P_{cap} increases. As water moves across the membrane, there is typically an increase in the Pᵢₛ and, because of the stress on the membrane, there is an increase in the efflux of sodium and plasma proteins raising

$$\begin{matrix} \frac{d {V}_{H 2 O}}{d t} = {k}_{p} [ ( {P}_{cap} - {P}_{is} ) - ( {Π}_{cap} - {Π}_{is} ) ] - {k}_{A} [ {Π}_{is} ] & ( 7 ) \\ {P}_{is} = η \cdot {V}_{H 2 O} & ( 8 ) \\ {Π}_{is} = ɛ \cdot {V}_{H 2 O} & ( 9 ) \\ {P}_{cap} = σ \cdot {P}_{LA} & ( 10 ) \\ \frac{d {V}_{H 2 O}}{d t} = {k}_{p} [ ( σ \cdot {P}_{LA} - η \cdot {V}_{H 2 O} ) - ( {Π}_{cap} - ɛ \cdot {V}_{H 2 O} ) ] - {k}_{A} \cdot ɛ \cdot {V}_{H 2 O} & ( 11 ) \\ \frac{d {V}_{H 2 O}}{d t} = {k}_{p} σ \cdot {P}_{LA} - {k}_{p} \cdot {Π}_{cap} - [ {k}_{p} \cdot ( η - ɛ ) + {k}_{A} \cdot ɛ ) ] \cdot {V}_{H 2 O} & ( 12 ) \\ \frac{d {V}_{H 2 O}}{d t} = {k}_{p} σ \cdot {P}_{LA} - {k}_{p} \cdot {Π}_{cap} - B \cdot {V}_{H 2 O} & ( 13 )\end{matrix}$$

Note that Equation 13 is that of a first order differential equation. If P_{LA}, the left atrial pressure or LAP, is described as a function of time and if the blood's oncotic pressure is assumed to be a constant, then Equation 13 may be solved as a convolution integral. The result is shown in Equation 14. Note that this solution is consistent with a first order lag filter. Equation 14 acts like a low-pass filter that “smoothes” the signal (rather like an averaging process.) Though it should be understood that the output of this smoothing process is a fluid volume value (V_{H20}) not a pressure value.

$${V}_{H 2 O} ( t ) = {{\int }_{- ∞}}^{∞} [ {k}_{p} σ \cdot {P}_{LA} ( τ ) - {k}_{p} \cdot {\prod }_{cap} ] \cdot exp ( - B \cdot ( t - τ ) d τ \qquad ( 14 )$$

The time constant for Equation 15 is represented in Equation 15.

$$Time Constant = \frac{1}{B} = \frac{1}{{k}_{p} \cdot ( η - ɛ ) + {k}_{A} \cdot ɛ} \qquad ( 15 )$$

Note further that Equation 14 may be approximated by difference Equation 16. Solving Equation 14 as a difference Equation 16 (below) has many advantages, e.g., it lends itself to digital solution techniques and it allows for non-linearities to be taken into account. For instance, the alveolar oncotic pressure may be a non-linear function of the P_{LA}. Any combination of non-linear relations may be accounted for based on the digital solution of the equations.

$${V}_{H 2 O} ( n ) = \frac{{V}_{H 2 O} ( n - 1 ) + ( {k}_{p} σ \cdot {P}_{LA} ( τ ) - {k}_{p} \cdot {Π}_{cap} ) Δ t}{1 + ( {k}_{p} \cdot ( η - ɛ ) + {k}_{A} \cdot ɛ ) Δ t} \qquad ( 16 )$$

The model represented by the forgoing equations and analysis suggests that transients in pressure will be “filtered out” when using the model. Only sustained upward trends in the P_{LA} will result in significant accumulation in lung water. The converse is also true: once lung water has accumulated, it may take some time for the active transport process (related by kₐ) to the passive transport process to drive the lung water back into the blood water.

FIG. 3 schematically illustrates the overall model. Briefly, within a patient, the lungs 100 respond to changes in LAP 102, resulting in changes in the amount of lung water (i.e. changes on pulmonary fluid volumes.) Likewise, a lung water predictor model 106 (properly calibrated) responds to changes in LAP samples 108 detected by a pacer/ICD, resulting in changes in the predicted amount of lung water 110.

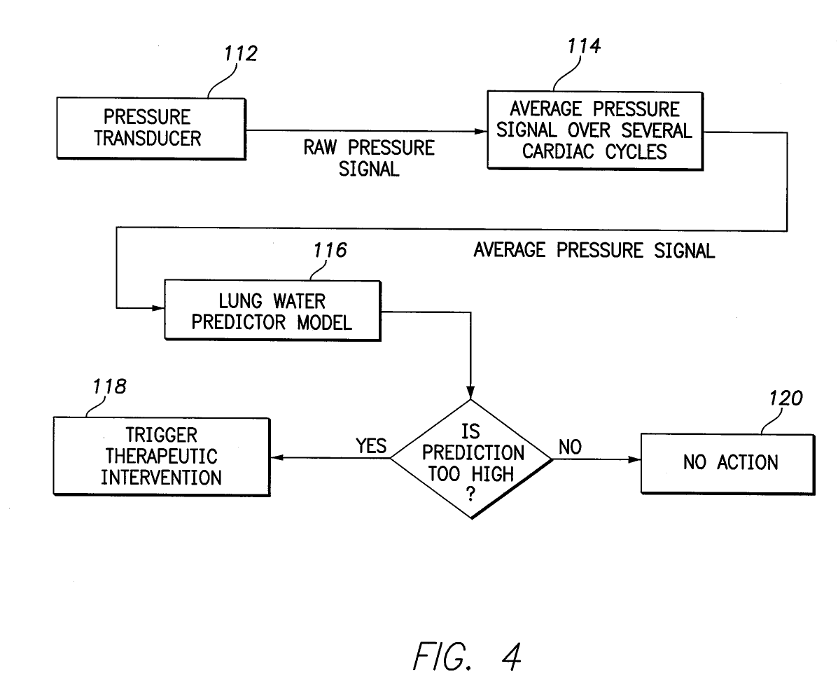

FIG. 4 provides a high-level summary of a method for exploiting the model. At step 112, a pressure transducer is employed to detect raw LAP signals, which are then averaged over several cardiac cycles at step 114. The average pressure signal is then applied to the lung water predictor model at step 116 to generate an output signal indicative of the predicted amount of lung water (i.e. output 110 of FIG. 3.) If the predicted value exceeds a threshold, therapeutic intervention is triggered, at step 118, such as administration of diuretics. Otherwise, at step 120, no action is taken. Although not shown, processing returns to step 112 to detect additional pressure values. Steps 112-118/120 are repeated in a loop within the patient to periodically detect or predict clinically-significant pulmonary fluid accumulations.

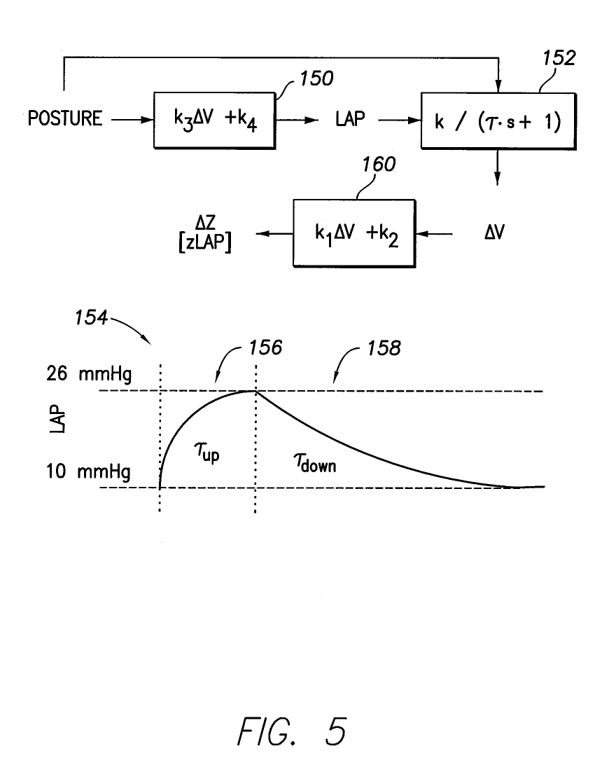

FIG. 5 provides further information regarding a “transfer function” relationship between LAP and pulmonary fluids, which additionally incorporates changes in posture. Briefly, posture may be regarded as affecting LAP via a first transfer function 150 represented by k₃ ΔV+k₄, where k₃ and k₄ are constants that can vary from patient to patient. That is, transfer function 150 represents a predictive model that relates changes in posture to changes in LAP. In turn, LAP can be regarded as affecting pulmonary fluids (represented via a change or “delta” in fluids ΔV) via a second transfer function k/(τ·s+1) where k is another constant that can vary from patient to patient. That is, transfer function 152 represents a predictive model that relates changes in LAP and posture to changes in pulmonary fluids. The parameter τ is a nonlinear transfer function time parameter represented by a “τᵤₚ” value indicative of an exponential rate of change while LAP is increasing following a change in posture to a supine posture and a “τ_{down}” value indicative of an exponential rate of change while LAP is decreasing following a change in posture to a standing posture. Graph 154 illustrates exemplary changes in LAP as a result of changes in posture. During a first interval 156 following a change in posture from standing to supine, LAP increases generally exponentially, subject to exponential time constant τᵤₚ from a relatively low LAP value of 10 mmHg to a high value of 26 mmHg. (These values are, of course, merely exemplary.) During a second interval 158 following a change in posture from supine to standing, LAP decreases generally exponentially subject to exponential time constant τ_{down}.

Finally, ΔV can be regarded as affecting cardiogenic impedance (ΔZ) [at least along certain vectors through the heart] via a third transfer function k₁ ΔV−k₂ where k₁ and k₂ are also constants that can vary from patient to patient. That is, transfer function 154 represents a predictive model that relates changes in pulmonary fluids to changes in Z or in zLAP, the latter of which is a value derived from certain cardiogenic impedance signals. It is referred to as zLAP as it is sometimes used as a proxy for LAP in at least some circumstances.

Techniques for detecting cardiogenic Z or zLAP are discussed, for example, in published U.S. Patent Application No. 2008/0262361 of Gutfinger et al., entitled “System and Method for Calibrating Cardiac Pressure Measurements Derived from Signals Detected by an Implantable Medical Device.” See, also, U.S. patent application Ser. Nos. 11/558,101, 11/557,851, 11/557,870, 11/557,882 and 11/558,088, each entitled “Systems and Methods to Monitor and Treat Heart Failure Conditions”, of Panescu et al. See, also, U.S. patent application Ser. No. 11/558,194, by Panescu et al., entitled “Closed-Loop Adaptive Adjustment of Pacing Therapy based on Cardiogenic Impedance Signals Detected by an Implantable Medical Device.” Other techniques for calibrating impedance-based techniques are set forth in: U.S. Pat. No. 7,794,404 of Panescu et al., entitled “System and Method for Estimating Cardiac Pressure Using Parameters Derived from Impedance Signals Detected by an Implantable Medical Device.” See, also, U.S. patent application Ser. No. 11/779,350, by Wenzel et al., filed Jul. 18, 2007, entitled “System and Method for Estimating Cardiac Pressure based on Cardiac Electrical Conduction Delays using an Implantable Medical Device,” now U.S. Published Patent Application 2009/0018597.

Thus FIG. 5 summarizes an overall predictive model that relates posture and LAP to pulmonary fluids, which is exploited to detect clinically-significant pulmonary fluid accumulation.

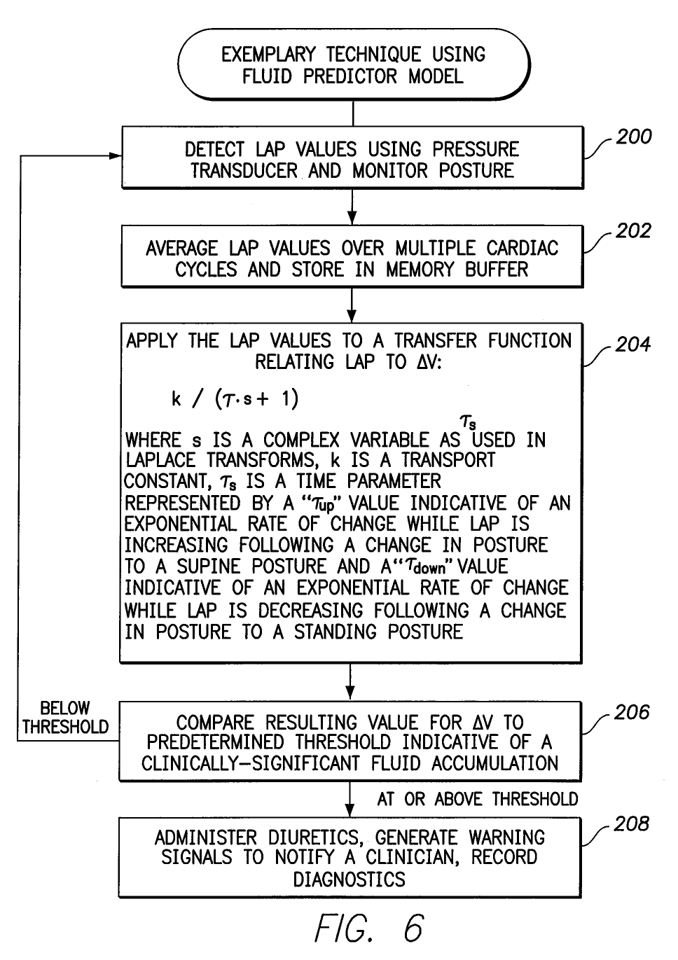

FIG. 6 provides a more detail example of the predictor model-based technique of FIG. 4 for detecting clinically-significant pulmonary fluid accumulations. Beginning at step 200, a pacer/ICD detects LAP values using a suitable pressure transducer and monitors changes in posture. See, e.g., posture detection techniques described in U.S. Pat. No. 6,658,292 of Kroll et al., entitled “Detection of Patient's Position and Activity Status Using 3D Accelerometer-Based Position Sensor.” See, also, U.S. Pat. No. 7,149,579, of Koh et al., entitled “System and Method for Determining Patient Posture based on 3-D Trajectory Using an Implantable Medical Device.”

At step 202, the pacer/ICD averages the LAP values over multiple cardiac cycles and stores the values in a memory buffer. At step 204, the pacer/ICD applies the averaged LAP values transfer function relating LAP to ΔV:

LAP→k/(τ·s+1)→ΔV (17)

where s is a complex variable as used in Laplace transforms, k is the aforementioned transport constant and τ is a time parameter that can be represented by the “τᵤₚ” value (indicative of an exponential rate of change while LAP is increasing following a change in posture to supine) and the “τ_{down}” value (indicative of an exponential rate of change while LAP is decreasing following a change in posture to standing.) Techniques will be described below for calibrating these values.

Thus, at step 204, the latest averaged LAP values are applied to the transfer function of Equation 17 to generate “predicted” or “smoothed” ΔV values, which are representative of fluid accumulations. It should be understood that this transfer function provides only an approximation of pulmonary fluid volume, based on LAP and binary posture (standing vs. supine.) In other implementations, additional factors might be taken into account, such as sodium ion levels, patient cardiac condition, patient weight, currently prescribed drugs and their effects, and more precise posture information.

At step 206, the latest value for ΔV is compared to a predetermined fluid volume threshold indicative of a clinically-significant fluid accumulation, such as 18 mmHg. Assuming that the value remains at or below the fluid volume threshold, then steps 200-206 are repeated. If the value exceeds the threshold, then, at step 208, the pacer/ICD administers diuretics, generates warning signals to notify a clinician, records diagnostics, etc., as already described. (As can be appreciated, some number of values of LAP might need to be processed using the transfer function of Equation 17 before reliable values for ΔV are obtained for comparison purposes.)

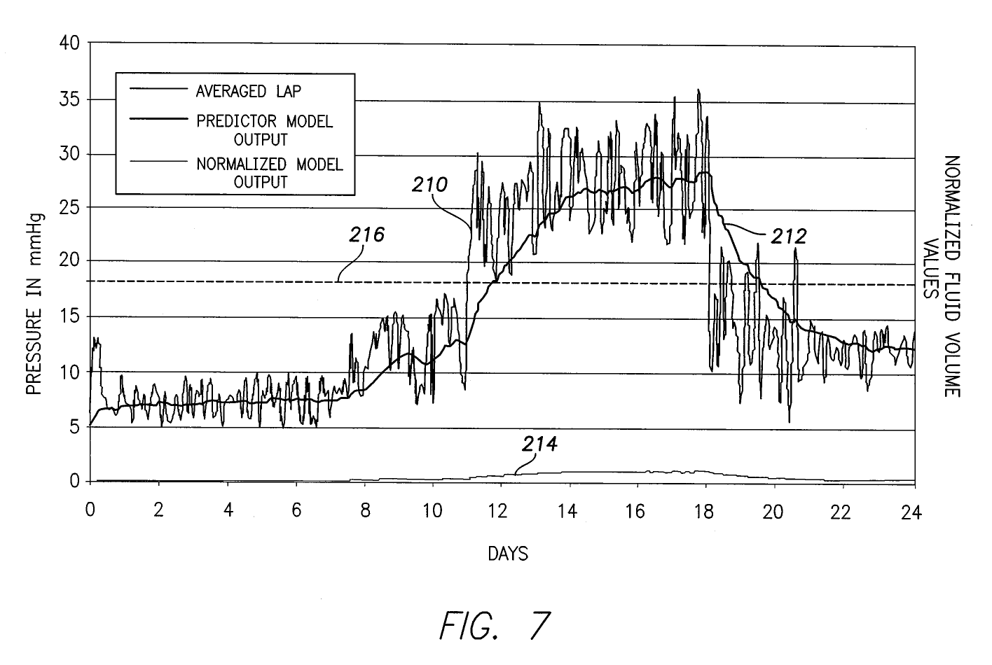

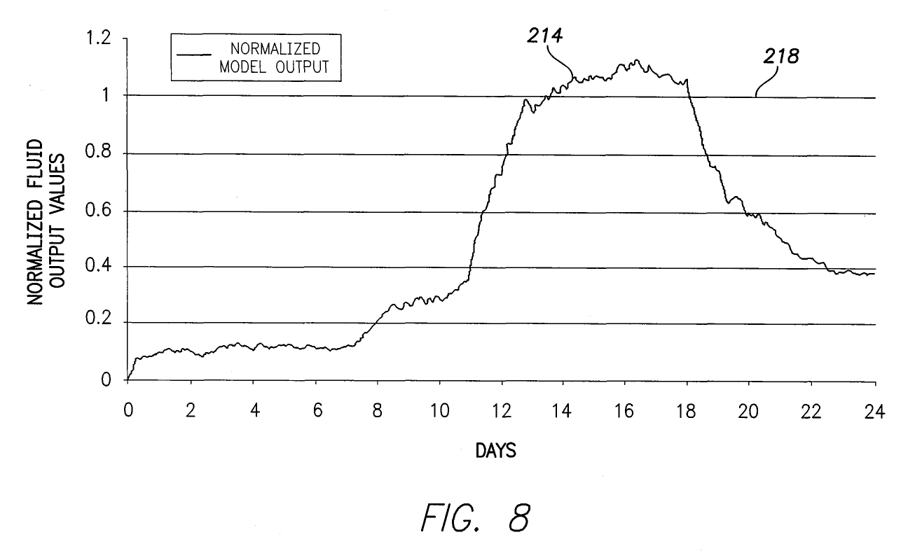

FIG. 7 illustrates exemplary averaged LAP values 210 (generated at step 202 of FIG. 6) and resulting predictor model output values 212 (i.e. predicted ΔV values generated at step 204 of FIG. 6), as well as normalized output values 214. Also shown is a pressure threshold 216, which can be applied to the non-normalized output values. In this example, the threshold for use with non-normalized output values is set to 18 mmHg. In practice, it is preferable to normalize the output for use with a detection threshold set to 1.0. This is illustrated in FIG. 8 wherein the predictor model output values 214 have been normalized for comparison against a threshold 218 set to of 1.0. As can be seen, the predicator model output signal exceeds the threshold at about Day 13, indicative of a clinically-significant pulmonary fluid accumulation. Note, in particular, that the predictor model output signals are relatively smooth, indicating that transients in LAP are mostly eliminated thus reducing or preventing false positive detections of pulmonary fluid accumulations. Indeed, as already explained, the predictor model essentially operates as a low-pass filter to smooth LAP values to eliminate transients to allow detection of clinically-significant increases in pulmonary fluids.

### Calibration Technique

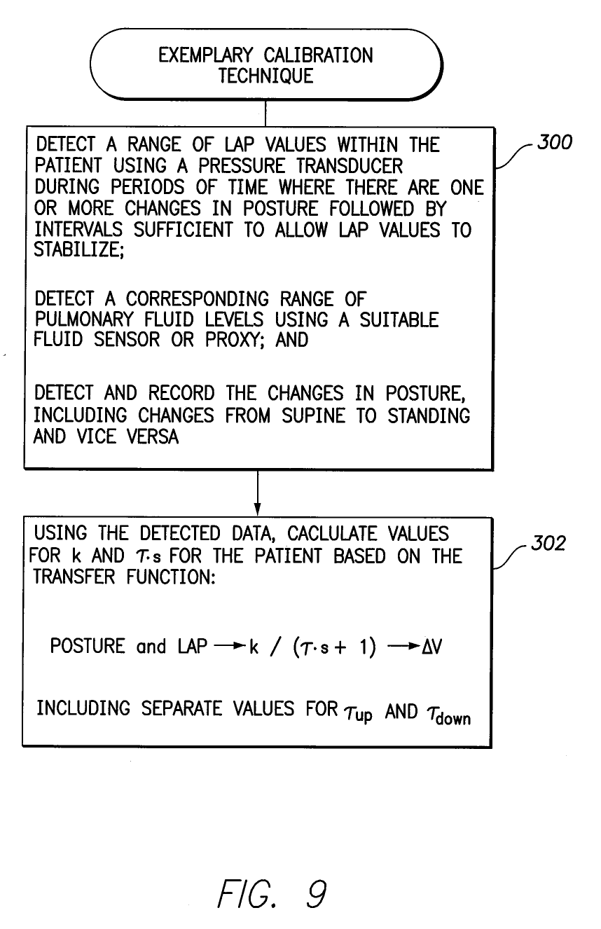

FIG. 9 illustrates a technique for calibrating values for k and τ for a patient for use in the transfer function of step 204 of FIG. 6. Beginning at step 300, the pacer/ICD detects a range of LAP values within the patient using pressure transducer 13 (of FIG. 1.) The LAP values are collected during periods of time when there are one or more changes in posture followed by intervals sufficient to allow LAP values to stabilize. For example, data may be collected over a twenty-four hour period so as to obtain values during the day while the patient is standing or walking (e.g. active) and to obtain values at night while the patient is inactive and supine (e.g. sleeping.) Also at step 300, the pacer/ICD additionally detects a corresponding a range of pulmonary fluid levels (V) using a suitable fluid sensor or proxy, while also detecting and recording the changes in posture, including changes from supine to standing and vice versa. Insofar as detecting values for V is concerned, if the device is equipped to detect pulmonary artery pressure, such pressure values can be used in at least some patients as a proxy for pulmonary fluid levels. For pulmonary artery pressure sensors, see, for example, U.S. Pat. No. 7,195,594 to Eigler, et al., entitled “Method for Minimally Invasive Calibration of Implanted Pressure Transducers” and U.S. Pat. No. 7,621,879 also of Eigler, et al., entitled “System for Calibrating Implanted Sensors.” See, also, U.S. Pat. No. 7,460,909 of Koh, et al., entitled “Implantable Device for Monitoring Hemodynamic Profiles.” Transthoracic impedance may also be used as a proxy or pulmonary fluid levels.

At step 302, the pacer/ICD then uses the detected data to calculate values for k and τ for the patient based on the transfer function of step 204 of FIG. 6 wherein posture and LAP→k/(τ·s+1)→ΔV, including calculating separate values for τᵤₚ and τ_{down} (which take into account the directional change in posture.) Otherwise conventional data processing techniques may be employed to determine preferred or optimal values for k, τᵤₚ and τ_{down} for the patient based on control system and feedback system techniques. In this regard, LAP and V data collected following a change in posture from supine to standing can be used to detect a suitable value for τᵤₚ (wherein sufficient time has elapsed for LAP and V to reach stable values); whereas LAP and V data collected following a change in posture from standing to supine can be used to detect a suitable value for τ_{down} (wherein sufficient time has elapsed for LAP and V to again reach stable values). Still further, these preferred or optimal values can be used to implement or approximate a low-pass filter using otherwise conventional filter calibration and configuration techniques.

What have been described are various techniques for detecting clinically-significant pulmonary fluid accumulations within the lungs of a patient. For the sake of completeness, a detailed description of an exemplary pacer/ICD for performing these techniques will now be provided. However, principles of invention may be implemented within other pacer/ICD implementations or within other implantable devices such as stand-alone monitoring devices, CRT devices or CRT-D devices. (A CRT-D is a cardiac resynchronization therapy device with defibrillation capability.) Furthermore, although examples described herein involve processing of LAP data by the implanted device itself, some operations may be performed using an external device, such as a bedside monitor, device programmer, computer server or other external system. For example, LAP parameters might be transmitted to the external device, which processes the data to detect a clinically-significant pulmonary fluid accumulation. Processing by the implanted device itself is preferred as that allows the device to detect a clinically-significant pulmonary fluid accumulation more promptly and to take appropriate action.

Note also that the technique described herein may be selectively combined with, or corroborated by, other pulmonary fluid monitoring techniques, where appropriate. See, for example, U.S. patent application Ser. No. 12/210,848, of Bornzin et al., filed Sep. 15, 2008, entitled “System and Method for Monitoring Thoracic Fluid Levels Based on Impedance Using an Implantable Medical Device,” now U.S. Published Patent Application 2010/0069778.

### PAP-Based System

FIG. 10 broadly summarizes a general technique for detecting a clinically-significant pulmonary fluid accumulation based on PAP that may be exploited by the pacer/ICD of FIG. 1 or other suitably equipped systems. Beginning at step 350, the pacer/ICD detects values representative of PAP within the patient and then, at step 352, tracks changes in PAP over time indicative of possible pulmonary fluid accumulation and determines whether the changes in PAP values are both sufficiently elevated and sufficiently prolonged to warrant clinical intervention. That is, at least part of the determination of whether the pulmonary fluid accumulation is clinically-significant is made based on an elevation in PAP in combination with the duration with which PAP remains elevated, thereby avoiding false positive event detections due to PAP transients or other factors. Predictor models of the type discussed above can modified (without undue effort or experimentation) to process PAP values to estimate pulmonary fluid volumes to make this determination. At step 354, the pacer/ICD then administers diuretics, generates warning signals to notify the clinician, records diagnostics and/or controls other device functions in response to a determination that clinical intervention is warranted. Note that in, at least many cases, PAP can be used as a surrogate for LAP. Alternatively, one parameter can often be used to supplement or corroborate the other.

### Exemplary Pacer/ICD

FIG. 11 provides a simplified block diagram of the pacer/ICD, which is a dual-chamber stimulation device capable of treating both fast and slow arrhythmias with stimulation therapy, including cardioversion, defibrillation, and pacing stimulation, as well as capable of performing the pulmonary fluid monitoring functions described above. To provide atrial chamber pacing stimulation and sensing, pacer/ICD 10 is shown in electrical communication with a heart 512 by way of a left atrial lead 520 having an atrial tip electrode 522 and an atrial ring electrode 523 implanted in the atrial appendage. Pacer/ICD 10 is also in electrical communication with the heart by way of a right ventricular lead 530 having, in this embodiment, a ventricular tip electrode 532, a right ventricular ring electrode 534, a right ventricular (RV) coil electrode 536, and a superior vena cava (SVC) coil electrode 538. Typically, the right ventricular lead 530 is transvenously inserted into the heart so as to place the RV coil electrode 536 in the right ventricular apex, and the SVC coil electrode 538 in the superior vena cava. Accordingly, the right ventricular lead is capable of receiving cardiac signals, and delivering stimulation in the form of pacing and shock therapy to the right ventricle.

To sense left atrial and ventricular cardiac signals and to provide left chamber pacing therapy, pacer/ICD 10 is coupled to a “coronary sinus” lead 524 designed for placement in the “coronary sinus region” via the coronary sinus os for positioning a distal electrode adjacent to the left ventricle and/or additional electrode(s) adjacent to the left atrium. As used herein, the phrase “coronary sinus region” refers to the vasculature of the left ventricle, including any portion of the coronary sinus, great cardiac vein, left marginal vein, left posterior ventricular vein, middle cardiac vein, and/or small cardiac vein or any other cardiac vein accessible by the coronary sinus. Accordingly, an exemplary coronary sinus lead 524 is designed to receive atrial and ventricular cardiac signals and to deliver left ventricular pacing therapy using at least a left ventricular tip electrode 526, left atrial pacing therapy using at least a left atrial ring electrode 527, and shocking therapy using at least a left atrial coil electrode 528. With this configuration, biventricular pacing can be performed. An LAP transducer is shown mounted adjacent the left atria along lead 524. This location is merely illustrative. The actual location of the LAP transducer may differ. Again, see the patent documents cited above.

Although only three leads are shown in FIG. 11, it should also be understood that additional stimulation leads (with one or more pacing, sensing and/or shocking electrodes) might be used in order to efficiently and effectively provide pacing stimulation to the left side of the heart or atrial cardioversion and/or defibrillation.

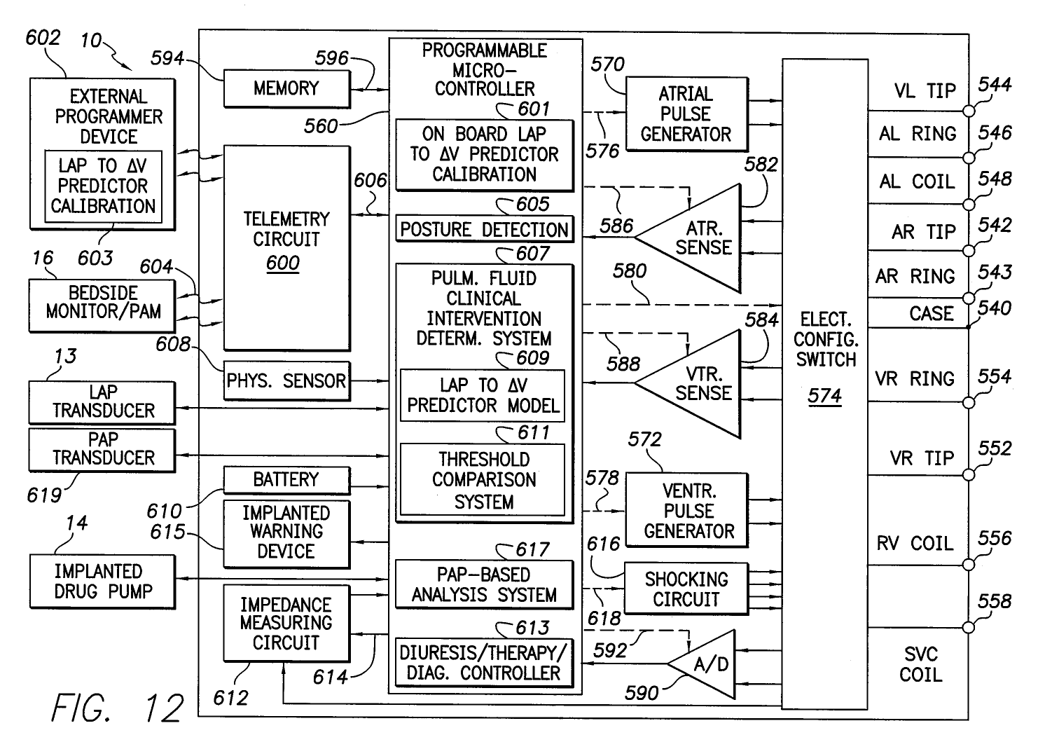

A simplified block diagram of internal components of pacer/ICD 10 is shown in FIG. 12. While a particular pacer/ICD is shown, this is for illustration purposes only, and one of skill in the art could readily duplicate, eliminate or disable the appropriate circuitry in any desired combination to provide a device capable of treating the appropriate chamber(s) with cardioversion, defibrillation and pacing stimulation as well as providing for the aforementioned impedance-based functions.

The housing 540 for pacer/ICD 10, shown schematically in FIG. 12, is often referred to as the “can”, “case” or “case electrode” and may be programmably selected to act as the return electrode for all “unipolar” modes. The housing 540 may further be used as a return electrode alone or in combination with one or more of the coil electrodes, 528, 536 and 538, for shocking purposes. The housing 540 further includes a connector (not shown) having a plurality of terminals, 542, 543, 544, 546, 548, 552, 554, 556 and 558 (shown schematically and, for convenience, the names of the electrodes to which they are connected are shown next to the terminals). As such, to achieve right atrial sensing and pacing, the connector includes at least a right atrial tip terminal (A_{R} TIP) 542 adapted for connection to the atrial tip electrode 522 and a right atrial ring (A_{R} RING) electrode 543 adapted for connection to right atrial ring electrode 523. To achieve left chamber sensing, pacing and shocking, the connector includes at least a left ventricular tip terminal (V_{L} TIP) 544, a left atrial ring terminal (A_{L} RING) 546, and a left atrial shocking terminal (A_{L} COIL) 548, which are adapted for connection to the left ventricular ring electrode 526, the left atrial tip electrode 527, and the left atrial coil electrode 528, respectively. To support right chamber sensing, pacing and shocking, the connector further includes a right ventricular tip terminal (V_{R} TIP) 552, a right ventricular ring terminal (V_{R} RING) 554, a right ventricular shocking terminal (R_{V} COIL) 556, and an SVC shocking terminal (SVC COIL) 558, which are adapted for connection to the right ventricular tip electrode 532, right ventricular ring electrode 534, the RV coil electrode 536, and the SVC coil electrode 538, respectively. Although not shown, additional terminals may be needed for use with LAP transducer 13 and drug pump 14.

At the core of pacer/ICD 10 is a programmable microcontroller 560, which controls the various modes of stimulation therapy. As is well known in the art, the microcontroller 560 (also referred to herein as a control unit) typically includes a microprocessor, or equivalent control circuitry, designed specifically for controlling the delivery of stimulation therapy and may further include RAM or ROM memory, logic and timing circuitry, state machine circuitry, and I/O circuitry. Typically, the microcontroller 560 includes the ability to process or monitor input signals (data) as controlled by a program code stored in a designated block of memory. The details of the design and operation of the microcontroller 560 are not critical to the invention. Rather, any suitable microcontroller 560 may be used that carries out the functions described herein. The use of microprocessor-based control circuits for performing timing and data analysis functions are well known in the art.

As shown in FIG. 12, an atrial pulse generator 570 and a ventricular/impedance pulse generator 572 generate pacing stimulation pulses for delivery by the right atrial lead 520, the right ventricular lead 530, and/or the coronary sinus lead 524 via an electrode configuration switch 574. It is understood that in order to provide stimulation therapy in each of the four chambers of the heart, the atrial and ventricular pulse generators, 570 and 572, may include dedicated, independent pulse generators, multiplexed pulse generators or shared pulse generators. The pulse generators, 570 and 572, are controlled by the microcontroller 560 via appropriate control signals, 576 and 578, respectively, to trigger or inhibit the stimulation pulses.

The microcontroller 560 further includes timing control circuitry (not separately shown) used to control the timing of such stimulation pulses (e.g., pacing rate, atrio-ventricular (AV) delay, atrial interconduction (A-A) delay, or ventricular interconduction (V-V) delay, etc.) as well as to keep track of the timing of refractory periods, blanking intervals, noise detection windows, evoked response windows, alert intervals, marker channel timing, etc., which is well known in the art. Switch 574 includes a plurality of switches for connecting the desired electrodes to the appropriate I/O circuits, thereby providing complete electrode programmability. Accordingly, the switch 574, in response to a control signal 580 from the microcontroller 560, determines the polarity of the stimulation pulses (e.g., unipolar, bipolar, combipolar, etc.) by selectively closing the appropriate combination of switches (not shown) as is known in the art.

Atrial sensing circuits 582 and ventricular sensing circuits 584 may also be selectively coupled to the right atrial lead 520, coronary sinus lead 524, and the right ventricular lead 530, through the switch 574 for detecting the presence of cardiac activity in each of the four chambers of the heart. Accordingly, the atrial (ATR. SENSE) and ventricular (VTR. SENSE) sensing circuits, 582 and 584, may include dedicated sense amplifiers, multiplexed amplifiers or shared amplifiers. The switch 574 determines the “sensing polarity” of the cardiac signal by selectively closing the appropriate switches, as is also known in the art. In this way, the clinician may program the sensing polarity independent of the stimulation polarity. Each sensing circuit, 582 and 584, preferably employs one or more low power, precision amplifiers with programmable gain and/or automatic gain control, bandpass filtering, and a threshold detection circuit, as known in the art, to selectively sense the cardiac signal of interest. The automatic gain control enables pacer/ICD 10 to deal effectively with the difficult problem of sensing the low amplitude signal characteristics of atrial or ventricular fibrillation. The outputs of the atrial and ventricular sensing circuits, 582 and 584, are connected to the microcontroller 560 which, in turn, are able to trigger or inhibit the atrial and ventricular pulse generators, 570 and 572, respectively, in a demand fashion in response to the absence or presence of cardiac activity in the appropriate chambers of the heart.

For arrhythmia detection, pacer/ICD 10 utilizes the atrial and ventricular sensing circuits, 582 and 584, to sense cardiac signals to determine whether a rhythm is physiologic or pathologic. As used herein “sensing” is reserved for the noting of an electrical signal, and “detection” is the processing of these sensed signals and noting the presence of an arrhythmia. The timing intervals between sensed events (e.g., P-waves, R-waves, and depolarization signals associated with fibrillation which are sometimes referred to as “F-waves” or “Fib-waves”) are then classified by the microcontroller 560 by comparing them to a predefined rate zone limit (i.e., bradycardia, normal, atrial tachycardia, atrial fibrillation, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, antitachycardia pacing, cardioversion shocks or defibrillation shocks).

Cardiac signals are also applied to the inputs of an analog-to-digital (A/D) data acquisition system 590. The data acquisition system 590 is configured to acquire intracardiac electrogram signals, convert the raw analog data into a digital signal, and store the digital signals for later processing and/or telemetric transmission to an external device 602. The data acquisition system 590 is coupled to the right atrial lead 520, the coronary sinus lead 524, and the right ventricular lead 530 through the switch 574 to sample cardiac signals across any pair of desired electrodes. The microcontroller 560 is further coupled to a memory 594 by a suitable data/address bus 596, wherein the programmable operating parameters used by the microcontroller 560 are stored and modified, as required, in order to customize the operation of pacer/ICD 10 to suit the needs of a particular patient. Such operating parameters define, for example, pacing pulse amplitude or magnitude, pulse duration, electrode polarity, rate, sensitivity, automatic features, arrhythmia detection criteria, and the amplitude, waveshape and vector of each shocking pulse to be delivered to the patient's heart within each respective tier of therapy. Other pacing parameters include base rate, rest rate and circadian base rate.

Advantageously, the operating parameters of the implantable pacer/ICD 10 may be non-invasively programmed into the memory 594 through a telemetry circuit 600 in telemetric communication with the external device 602, such as a programmer, transtelephonic transceiver or a diagnostic system analyzer. The telemetry circuit 600 is activated by the microcontroller by a control signal 606. The telemetry circuit 600 advantageously allows intracardiac electrograms and status information relating to the operation of pacer/ICD 10 (as contained in the microcontroller 560 or memory 594) to be sent to the external device 602 through an established communication link 604. Pacer/ICD 10 further includes an accelerometer or other physiologic sensor 608, commonly referred to as a “rate-responsive” sensor because it is typically used to adjust pacing stimulation rate according to the exercise state of the patient. However, the physiological sensor 608 may further be used to detect changes in cardiac output, changes in the physiological condition of the heart, or diurnal changes in activity (e.g., detecting sleep and wake states) and to detect arousal from sleep. Additionally, sensor 608 could be equipped to detect pulmonary fluid levels or proxies for pulmonary fluid levels. Accordingly, the microcontroller 560 responds by adjusting the various pacing parameters (such as rate, AV Delay, V-V Delay, etc.) at which the atrial and ventricular pulse generators, 570 and 572, generate stimulation pulses. While shown as being included within pacer/ICD 10, it is to be understood that the physiologic sensor 608 may also be external to pacer/ICD 10, yet still be implanted within or carried by the patient. A common type of rate responsive sensor is an activity sensor incorporating an accelerometer or a piezoelectric crystal, which is mounted within the housing 540 of pacer/ICD 10. Other types of physiologic sensors are also known, for example, sensors that sense the oxygen content of blood, respiration rate and/or minute ventilation, pH of blood, ventricular gradient, pulmonary artery pressure, etc.

The pacer/ICD additionally includes a battery 610, which provides operating power to all of the circuits shown in FIG. 12. The battery 610 may vary depending on the capabilities of pacer/ICD 10. If the system only provides low voltage therapy, a lithium iodine or lithium copper fluoride cell may be utilized. For pacer/ICD 10, which employs shocking therapy, the battery 610 must be capable of operating at low current drains for long periods, and then be capable of providing high-current pulses (for capacitor charging) when the patient requires a shock pulse. The battery 610 must also have a predictable discharge characteristic so that elective replacement time can be detected. Accordingly, pacer/ICD 10 is preferably capable of high voltage therapy and appropriate batteries.

As further shown in FIG. 12, pacer/ICD 10 is shown as having an impedance measuring circuit 612, which is enabled by the microcontroller 560 via a control signal 614. Herein, impedance is primarily detected for use in evaluating thoracic and cardiogenic impedance for use in evaluating thoracic fluids. Other uses for an impedance measuring circuit include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring stroke volume; and detecting the opening of heart valves, etc. The impedance measuring circuit 612 is advantageously coupled to the switch 574 so that any desired electrode may be used.

In the case where pacer/ICD 10 is intended to operate as an implantable cardioverter/defibrillator (ICD) device, it detects the occurrence of an arrhythmia, and automatically applies an appropriate electrical shock therapy to the heart aimed at terminating the detected arrhythmia. To this end, the microcontroller 560 further controls a shocking circuit 616 by way of a control signal 618. The shocking circuit 616 generates shocking pulses of low (up to 0.5 joules), moderate (0.5-10 joules) or high energy (11 to 40 joules), as controlled by the microcontroller 560. Such shocking pulses are applied to the heart of the patient through at least two shocking electrodes, and as shown in this embodiment, selected from the left atrial coil electrode 528, the RV coil electrode 536, and/or the SVC coil electrode 538. The housing 540 may act as an active electrode in combination with the RV electrode 536, or as part of a split electrical vector using the SVC coil electrode 538 or the left atrial coil electrode 528 (i.e., using the RV electrode as a common electrode). Cardioversion shocks are generally considered to be of low to moderate energy level (so as to minimize pain felt by the patient), and/or synchronized with an R-wave and/or pertaining to the treatment of tachycardia. Defibrillation shocks are generally of moderate to high energy level (i.e., corresponding to thresholds in the range of 5-40 joules), delivered asynchronously (since R-waves may be too disorganized), and pertaining exclusively to the treatment of fibrillation. Accordingly, the microcontroller 560 is capable of controlling the synchronous or asynchronous delivery of the shocking pulses.

Microcontroller 560 also includes various components directed to implementing the aforementioned pulmonary fluid monitoring methods. More specifically, an on-board LAP to ΔV predictor calibration system 601 calibrates values for k and τ using techniques described above (FIG. 9) based on LAP values detected by LAP transducer 13 in combination with pulmonary fluid values (V) sensed by a suitable implantable pulmonary fluid detector, which might be part of the physiological sensor system 608 already described (or values obtained using an external fluid detection system and received via external device 602.) A posture detection system 605 is operative to detect changes in posture. Alternatively, calibration can be performed or controlled by suitable components of the external programmer, such as by predictor calibration system 603 of the programmer.

A pulmonary fluid clinical intervention determination system 607 is operative to track changes in LAP values over time indicative of possible pulmonary fluid accumulation within the patient and to determine whether the changes in LAP values are sufficiently elevated and prolonged to warrant clinical intervention. That is, the clinical intervention determination system determines whether there is a clinically-significant pulmonary fluid accumulation. To this end, the clinical intervention determination system uses an LAP to ΔV predictor model 609 (operative to perform techniques described above with reference to FIGS. 3-8.) A threshold comparison system 611 compares the output of the predictor model to detect a clinically-significant pulmonary fluid overload.

A diuresis/therapy/diagnostics controller 613 controls generation of diagnostic data and warning signals in response to a clinically-significant pulmonary fluid accumulation. Diagnostic data is stored within memory 594. Warning signals may be relayed to the patient via implanted warning device 615 or via bedside monitor/PAM 16. Controller 613 also controls and titrates the delivery of diuretics (or other appropriate therapies) using drug pump 14 as described above. In implementations where there is no drug pump, titration of diuretics is typically achieved by instead providing suitable instructions to the patient or caregiver via the bedside monitor (or other external device).

Additionally or alternatively, the device may include a PAP-based controller operative 617 to control or perform the steps of FIG. 10. As such, although not separately illustrated in FIG. 12, the microcontroller can contain components analogous to blocks 601-611 but adapted for use with PAP. PAP signals may be received from a PAP transducer 619 (i.e. PAP sensor, PAP detector or other suitable PAP meaurement device) implanted within the patient. Sensors for use in the pulmonary artery are discussed, for example, in U.S. Pat. No. 7,621,036 of Cros, et al., entitled “Method of Manufacturing Implantable Wireless Sensor for In Vivo Pressure Measurement” and in U.S. Published Patent Application 2006/0287602 of O'Brien et al., entitled “Implantable Wireless Sensor for In Vivo Pressure Measurement,” both assigned to CardioMems, Inc.

Note that to accommodate the LAP and PAP tranducers, additional connection terminals may be employed. Alternatively, wireless communication may be employed. If the transducer is equipped to receive power via electromagnetic induction, the implanteddevice may be additionally provided with suitable power transmission systems. In some cases, an external power delivery wand may be appropriate. For a discussion of power delivery wands, see U.S. patent application Ser. No. 11/267,665, filed Nov. 4, 2005, of Kil et al., entitled “System and Method for Measuring Cardiac Output via Thermal Dilution using an Implantable Medical Device with Thermistor Implanted In Right Ventricle.”

Depending upon the implementation, the various components of the microcontroller may be implemented as separate software modules or the modules may be combined to permit a single module to perform multiple functions. In addition, although shown as being components of the microcontroller, some or all of these components may be implemented separately from the microcontroller. The components can also exploit or comprise expert systems.

What have been described are various systems and methods for use with a pacer/ICD. However, principles of the invention may be exploiting using other implantable medical systems. Thus, while the invention has been described with reference to particular exemplary embodiments, modifications can be made thereto without departing from the scope of the invention.

What is claimed is:

1. A method for use with an implantable medical device for implant within a patient, the method comprising:
detecting values representative of left atrial pressure (LAP) within the patient;
tracking changes in LAP values over time indicative of possible pulmonary fluid accumulation within the patient and determining whether the changes in LAP values are sufficiently elevated and prolonged to warrant clinical intervention; and
controlling at least one device function in response to a determination that clinical intervention is warranted.

2. The method of claim 1 for use with a device having an LAP transducer and wherein detecting values representative of LAP within the patient includes detecting the LAP values using the LAP transducer.

3. The method of claim 1 wherein detecting values representative of LAP is performed to detect values in the range of once every minute to once every five hours.

4. The method of claim 1 wherein detecting values representative of LAP is performed to detect a set of values over a series of cardiac cycles and to then average the values.

5. The method of claim 1 wherein tracking changes in LAP values over time and determining whether the changes are sufficiently elevated and prolonged to warrant clinical intervention includes:
applying a transfer function to the LAP values to generate values representative of pulmonary fluid accumulation (ΔV); and
comparing the values representative of pulmonary fluid accumulation (ΔV) against fluid accumulation thresholds indicative of a clinically-significant sustained accumulations.

6. The method of claim 5 wherein the transfer function is represented by:
LAP→k/(τ·s+1)→ΔV
where τ is representative of one or more exponential time parameters and where k is a constant and s is a complex variable.

7. The method of claim 6 wherein τ is dependent on posture and is represented by at least a τᵤₚ value representative of an exponential rate of change while LAP is increasing following a change in posture to a supine posture and a τ_{down} value representative of an exponential rate of change while LAP is decreasing following a change in posture to a standing posture.

8. The method of claim 7 further including the preliminary step of determining values for k, τᵤₚ and τ_{down} for the patient by:
determining values for LAP and fluid accumulation (ΔV) for the patient following changes in posture from supine to standing and from standing to supine; and
determining values for k, τᵤₚ and τ_{down} for the patient based on the values of LAP and ΔV.

9. The method of claim 7 wherein the transfer function relating LAP values to ΔV is configured as a low-pass filter with filtering parameters corresponding to the transfer function and wherein applying the transfer function to the LAP values is performed by applying the low-pass filter to the LAP values to generate smoothed LAP values for comparison against a pressure threshold indicative of a clinically-significant sustained pressure.

10. The method of claim 9 wherein the pressure threshold is in the range of 18 to 22 mmHg.

11. The method of claim 1 wherein controlling at least one device function in response to a determination that clinical intervention is warranted includes titrating a dosage of diuretics.

12. The method of claim 1 wherein controlling at least one device function in response to a determination that clinical intervention is warranted includes generating warning signals.

13. The method of claim 1 further including generating diagnostic information representative of pulmonary fluid accumulation.

17. A system for use with an implantable medical device for implant within a patient, the system comprising:
a left atrial pressure (LAP) detector operative to detect values representative of LAP within the patient; and
a pulmonary fluid clinical intervention determination system operative to track changes in LAP values over time indicative of possible pulmonary fluid accumulation within the patient and to determine whether the changes in LAP values are sufficiently elevated and prolonged to warrant clinical intervention.

18. A system for use with an implantable medical device for implant within a patient, the system comprising:
means for detecting values representative of left atrial pressure (LAP) within the patient;
means for tracking changes in LAP values over time indicative of possible pulmonary fluid accumulation within the patient; and
means for determining whether the changes in LAP values are sufficiently elevated and prolonged to warrant clinical intervention.

19. A method for use with an implantable medical device for implant within a patient, the method comprising:
detecting values representative of pulmonary artery pressure (PAP) within the patient;
tracking changes in PAP values over time indicative of possible pulmonary fluid accumulation within the patient and determining whether the changes in PAP values are sufficiently elevated and prolonged to warrant clinical intervention; and
controlling at least one device function in response to a determination that clinical intervention is warranted.
